(19) United States
(12) Patent Application Publication
(10) Pub. No.: US 2018/0161559 A1
(43) Pub. Date: Jun. 14, 2018

(54) APPLICATOR WITH LIQUID MEDICINE

(71) Applicant: HOGY MEDICAL CO., LTD., Tokyo (JP)

(72) Inventors: Kazuo SEI, Tokyo (JP); Jiro KATO, Tokyo (JP); Shiro RIKITAKE, Kumamoto-shi (JP); Shinichi YAMAKAWA, Kumamoto-shi (JP)

(73) Assignee: HOGY MEDICAL CO., LTD., Tokyo (JP)

(21) Appl. No.: 15/578,550

(22) PCT Filed: Jun. 8, 2016
(86) PCT No.: PCT/JP2016/066998

§ 371 Date: Nov. 30, 2017

(30) Foreign Application Priority Data
Jun. 24, 2015 (JP) 2015-126489

(51) Int. Cl. A61M 35/00

(52) U.S. Cl. CPC A61M 35/003; B05C 17/00

(57) ABSTRACT

An applicator with a liquid medicine where even when an accidental external pressure is applied during transport or storage, there is no possibility that a liquid medicine storage member is opened and a liquid medicine is impregnated into an applying member so that the liquid medicine can be reliably held but, at the same time, the liquid medicine can be easily impregnated into the applying member when necessary. The applicator with a liquid medicine includes a storage member for storing an applying member, the storage member includes: a bottom film in which the applying member is stored; and a top film which closes the bottom film thus storing the applying member and a liquid medicine storage member, in which the liquid medicine is sealed, in the storage member, and the bottom film has an air chamber capable of storing at least the applying portion and a predetermined amount of air.

### TECHNICAL FIELD

The present invention relates to an applicator with a liquid medicine used for applying a liquid medicine such as an antiseptic solution to a surgical field, and more specifically to an applicator with a liquid medicine from which a liquid medicine can be applied to a surgical field by merely opening a storage member in which an applying member is stored.

### BACKGROUND ART

In performing surgical treatment, when a liquid medicine such as an antiseptic solution is applied to a surgical field, in general, the liquid medicine is applied to the surgical field in a state of being impregnated into a cotton swab, a cotton ball or the like. However, the liquid medicine is separately prepared and impregnated into a cotton swab or a cotton ball each time the liquid medicine is applied to the surgical field, and such operations make the treatment cumbersome. Accordingly, to reduce an operation burden imposed on a medical doctor or nurse, there is known an applicator with a liquid medicine where a cotton swab or a cotton ball which is impregnated with a liquid medicine in advance is sealed in a container.

For example, an applicator with a liquid medicine described in patent literature 1 discloses an applicator with a liquid medicine which is an applicator stick package which includes: an applicator stick storage portion for sealing an applicator stick having an applying member on a shaft portion thereof; and a liquid agent storage portion for sealing a liquid agent such that the liquid agent is disposed adjacently to the applying member of the applicator stick by way of a separation band, wherein with pressure on the liquid agent storage portion, the liquid agent passes through the separation band and is impregnated into the applying member.

According to such an applicator with a liquid medicine, with pressure on the liquid agent storage portion, the liquid agent is impregnated into the applying member through the separation band so that the applicator stick can be used by merely extracting the applicator stick from the applicator stick package. Accordingly, an operation of separately preparing and dispensing a liquid agent can be omitted so that a treatment operation can be simplified. Further, the condition of the applying member being constantly impregnated with a liquid agent is avoided so that the deterioration of the applying member and any change in components of the liquid agent can be reliably prevented.

### CITATION LIST

### Patent Literature

### Patent Literature 1: Japanese Patent Laid-Open No. 2003-292041

### SUMMARY OF INVENTION

### Technical Problem

However, in the applicator with a liquid medicine described in patent literature 1, with pressure on the liquid agent storage portion, the separation band ruptures so that the liquid medicine is impregnated into the applying member. Accordingly, strength of the separation band cannot be increased so that when an external pressure is accidentally applied to the liquid agent storage portion from the outside, the liquid agent storage portion easily ruptures. In view of the above, the applicator with a liquid medicine described in patent literature 1 has a problem in that significant attention is required in handling the applicator with a liquid medicine. There is also a problem in that when an applicator is large-sized or an amount of liquid medicine to be impregnated into the applicator is increased, it is difficult to cause the whole amount of liquid medicine to be impregnated into the applicator, and the liquid medicine erroneously adheres to the applicator stick.

The present invention has been made to solve the above-mentioned problems, and an object of the present invention is to provide an applicator with a liquid medicine where even when an accidental external pressure is applied to the applicator with a liquid medicine, there is no possibility that a liquid medicine storage member is opened and a liquid medicine is impregnated into an applying member so that the liquid medicine can be reliably held but, at the same time, the liquid medicine can be easily and efficiently impregnated into the applying member when necessary.

### Solution to Problem

To solve the above-mentioned problem, an applicator with a liquid medicine according to the present invention is characterized in that the applicator includes: an applying member having a handle portion extending in a longitudinal direction and an applying portion mounted on one end of the handle portion and capable of being impregnated with a liquid medicine; and a storage member for storing the applying member, wherein the storage member includes: a bottom film in which the applying member is stored; and a top film which closes the bottom film thus storing the applying member and a liquid medicine storage member, in which the liquid medicine is sealed, in the storage member, and the bottom film has an air chamber which is capable of storing at least the applying portion and a predetermined amount of air.

Further, in the applicator with a liquid medicine according to the present invention, the bottom film preferably has a handle portion storage chamber which is made to communicate with the air chamber and stores the other end side of the handle portion.

Further, in the applicator with a liquid medicine according to the present invention, the liquid medicine storage member is preferably mounted on the top film, and the top film preferably has a point seal portion which is adhered to a portion near the handle portion storage chamber and is peelable.

Further, in the applicator with a liquid medicine according to the present invention, it is preferable that the liquid medicine storage member be disposed on the top film along an extending direction of the handle portion, the other end side of the liquid medicine storage member adhered to the top film, and an opening portion capable of being easily opened formed on one end side of the liquid medicine storage member.

Further, in the applicator with a liquid medicine according to the present invention, the top film is preferably adhered to the bottom film along an outer edge of the bottom film, and is preferably adhered to the bottom film such that the top film is not easily peelable on one end side of the bottom film.

Further, in the applicator with a liquid medicine according to the present invention, the top film is preferably adhered to the bottom film along the outer edge of the bottom film, and the top film is preferably adhered to the other end side of the bottom film by way of an air removal portion which allows the top film to be easily peeled away from the bottom film.

Still further, in the applicator with a liquid medicine according to the present invention, the air removal portion is preferably formed of a weak seal portion formed in an approximately V shape.

### Advantageous Effects of Invention

According to the present invention, the storage member includes: the bottom film in which the applying member is stored; and the top film which closes the bottom film thus storing the applying member and the liquid medicine storage member, in which the liquid medicine is sealed, in the storage member, and the bottom film has the air chamber which is capable of storing at least the applying portion and a predetermined amount of air. Accordingly, even when an accidental external pressure is applied to the storage member during transport, storage or the like, air sealed in the air chamber acts as a cushion so that it is possible to prevent that the liquid medicine storage member is opened and the liquid medicine sealed in the liquid medicine storage member leaks.

Further, according to the present invention, the air chamber has the handle portion storage chamber which is made to communicate with the air chamber and stores the other end side of the handle portion. Accordingly, by appropriately adjusting a volume of the handle portion storage chamber, a volume of the air chamber is adjusted so that an air pressure of air to be stored in the air chamber can be adjusted.

Further, according to the present invention, the liquid medicine storage member is mounted on the top film, and the top film has the point seal portion which is adhered to a portion near the handle portion storage chamber and is peelable. Accordingly, when the liquid medicine is impregnated into the applying portion, the point seal portion is peeled away so as to increase a volume of the air chamber whereby a cushioning action caused by air is eliminated. As a result, the liquid medicine storage member can be easily opened so that the liquid medicine can be impregnated into the applying portion.

Further, according to the present invention, the liquid medicine storage member is disposed on the top film along an extending direction of the handle portion, the other end side of the liquid medicine storage member being adhered to the top film, and the opening portion capable of being easily opened formed on one end side of the liquid medicine storage member. Accordingly, the liquid medicine storage member can be reliably mounted on the top film and, at the same time, the opening portion is positioned near the applying portion and hence, the liquid medicine can be efficiently impregnated into the applying portion.

Further, according to the present invention, the top film is adhered to the bottom film along an outer edge of the bottom film, and is adhered to the bottom film such that the top film is not easily peelable on one end side of the bottom film. Accordingly, in causing the liquid medicine to be impregnated into the applying member, it is possible to prevent the liquid medicine from leaking to the outside of the storage container. Further, after the impregnation of the liquid medicine is completed, in extracting the applying member, even when the liquid medicine is not completely impregnated into the applying member and some liquid medicine remains, it is possible to prevent the residual liquid medicine from spreading to the outside of the storage container or dripping from the storage container. Further, the applying member can be extracted without completely separating by peeling the top film from the bottom film. As described above, the top film is adhered to the bottom film at one end of the bottom film in a non-peelable manner. Accordingly, even when the liquid medicine is not completely impregnated into the applying portion and remains in the air chamber, there is no possibility that the liquid medicine spreads to the outside or drips from the storage container. As a result, the remaining liquid medicine can be held in the air chamber and, at the same time, there is no possibility that the top film is completely separated from the bottom film so that a disposal operation can be simplified in the disposal of the storage member.

Further, according to the present invention, the top film is adhered to the bottom film along the outer edge of the bottom film, and the top film is adhered to the other end side of the bottom film by way of the air removal portion which allows easy peeling of the top film. Accordingly, before the liquid medicine storage member is opened, the top film is slightly peeled away so as to remove air in the inside of the storage container through the air removal portion. With such an operation, a cushioning action caused by air can be further reduced so that the liquid medicine storage member can be reliably opened.

Further, according to the present invention, the air removal portion is formed of a weak seal portion formed in an approximately V shape. Accordingly, the air removal portion can be easily formed.

### BRIEF DESCRIPTION OF DRAWINGS

FIG. 1 is a top plan view of an applicator with a liquid medicine according to an embodiment of the present invention.

FIG. 2 is a bottom view of the applicator with a liquid medicine according to the embodiment of the present invention.

FIG. 3 is an exploded view of the applicator with a liquid medicine according to the embodiment of the present invention.

FIG. 4 is a view showing a procedure for using the applicator with a liquid medicine according to the embodiment of the present invention.

FIG. 5 is a view showing the procedure for using the applicator with a liquid medicine according to the embodiment of the present invention.

FIG. 6 is a view showing the procedure for using the applicator with a liquid medicine according to the embodiment of the present invention.

FIG. 7 is a view showing the procedure for using the applicator with a liquid medicine according to the embodiment of the present invention.

FIG. 8 is a view showing the procedure for using the applicator with a liquid medicine according to the embodiment of the present invention.

### DESCRIPTION OF EMBODIMENT

Hereinafter, an applicator with a liquid medicine according to the present invention is described with reference to drawings. The embodiment described hereinafter does not limit the invention according to respective claims. Further, it is not necessarily the case that all combinations of features described in the embodiment are indispensable as a solving means of the invention.

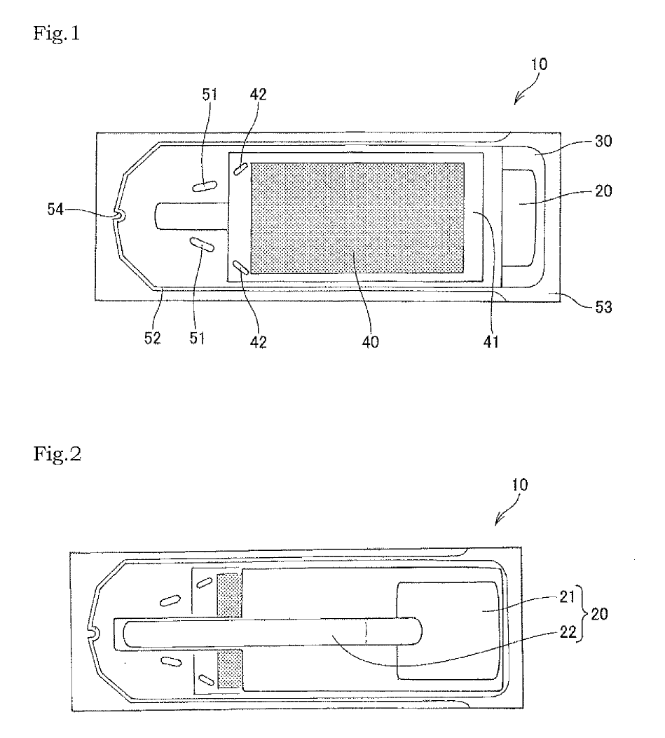

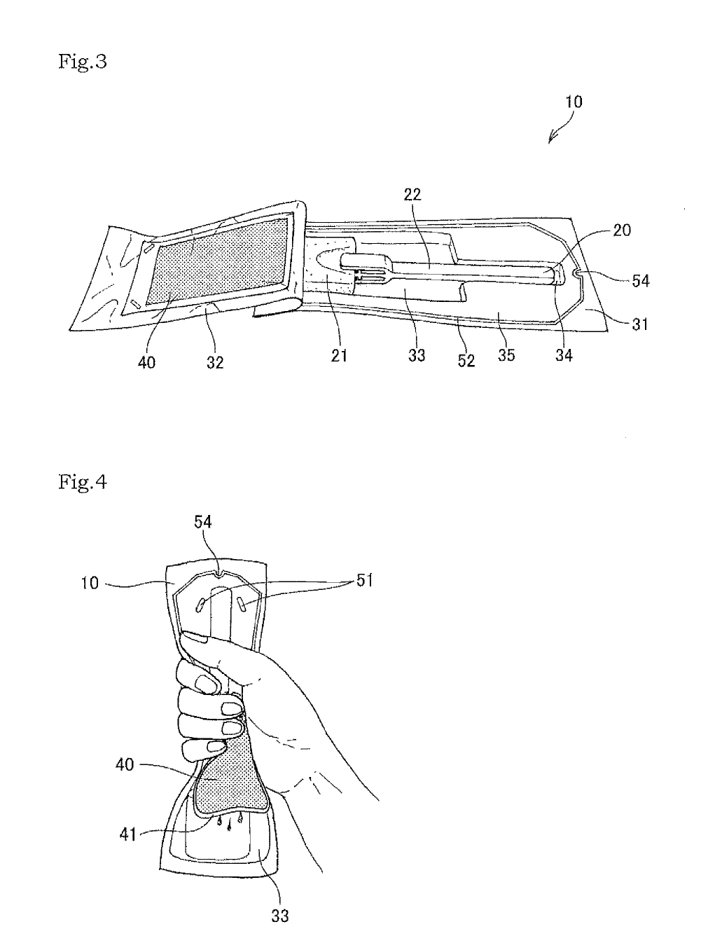

FIG. 1 is a top plan view of the applicator with a liquid medicine according to the embodiment of the present invention, FIG. 2 is a bottom view of the applicator with a liquid medicine according to the embodiment of the present invention, FIG. 3 is an exploded view of the applicator with a liquid medicine according to the embodiment of the present invention, and FIG. 4 to FIG. 8 are views showing a procedure for using the applicator with a liquid medicine according to the embodiment of the present invention. In the description made hereinafter, the description is made assuming that, in the extending direction of the handle portion 22, a side, on which an applying portion 21 is mounted on the handle portion 22, is one end side, and a side disposed on a side opposite to one side is the other end side.

As shown in FIG. 1 and FIG. 2, the applicator with a liquid medicine 10 according to this embodiment includes: an applying member 20 having the handle portion 22 extending in the longitudinal direction and the applying portion 21 mounted on one end of the handle portion 22; and a storage member 30 for storing the applying member 20.

The handle portion 22 is a rod-like member made of a synthetic resin or the like, for example, and the applying portion 21 is formed using a material such as a sponge or cotton, for example, which can be impregnated with a liquid medicine. Further, to be impregnated with and hold a liquid medicine, the applying portion 21 is preferably formed of a member having favorable liquid absorbency and favorable liquid-holding ability, for example. Polyurethane, cellulose or the like is preferably used as a material for forming the applying portion 21.

As shown in FIG. 3, the storage member 30 includes a bottom film 31 in which the applying member 20 is stored, and a top film 32 which closes the bottom film 31. Further, a liquid medicine storage member 40, in which a liquid medicine 43 is sealed, is adhered to the top film 32. As described above, the liquid medicine storage member 40 is adhered to the top film 32 and hence, an opening portion 41 of the liquid medicine storage portion 40 can be disposed at a desirable position such as a position near the applying portion 21, for example. Further, when the top film 32 is peeled away from the bottom film 31, the liquid medicine storage member 40 is separated from the bottom film 31 together with the top film 32 and hence, there is no possibility that the liquid medicine storage member 40 obstructs the extraction of the applying member 20 stored in the bottom film 31. Still further, a position where the top film 32 and the liquid medicine storage member 40 are adhered to each other is disposed on the other end side and hence, when the top film 32 is peeled away from the bottom film 31, the top film 32 and the liquid medicine storage member 40 can be integrally separated from the bottom film 31 from the other end side. Accordingly, a situation does not occur where the top film 32 and the bottom film 31 are separated from each other but the liquid medicine storage member 40 and the bottom film 31 are not separated from each other. As a result, there is no possibility that the liquid medicine storage member 40 obstructs the extraction of the applying member 20.

The bottom film 31 includes: a flat sheet portion 35; and an air chamber 33 and a handle portion storage chamber 34 each of which is formed on the sheet portion 35 in a recessed shape. The air chamber 33 is formed with a volume which allows the storage of at least the applying portion 21 and a predetermined amount of air. As described above, the predetermined amount of air is sealed in the air chamber 33 so that even when an external pressure is accidentally applied to the applicator with a liquid medicine 10 during transport, storage or the like, due to a cushioning effect caused by air, it is possible to prevent the liquid medicine storage member 40 from being opened.

Further, the handle portion storage chamber 34 is formed so as to store the other end side of the handle portion 22 such that the handle portion storage chamber 34 is made to communicate with the air chamber 33. The handle portion storage chamber 34 is formed with a width smaller than a width of the air chamber 33. To be more specific, the handle portion storage chamber 34 is formed to have a width which is approximately 30% of the width of the air chamber 33.

The top film 32 is a sheet-like member. As shown in FIG. 1 and FIG. 3, the liquid medicine storage member 40 is adhered to a surface of the top film 32, which opposedly faces the applying member 20, along the extending direction of the handle portion 22. The liquid medicine storage member 40 is adhered to the other end side of the top film 32 by means of liquid medicine storage member sealing portions 42. As described above, the liquid medicine storage member 40 is adhered to the other end side of the top film 32 and hence, when the top film 32 is peeled away from the other end side, the liquid medicine storage member 40 is separated from the bottom film 31 as an integral body with the top film 32. Further, the liquid medicine storage member 40 is formed such that an opening portion 41, where the liquid medicine storage member 40 is weakly sealed in a V shape, is formed on the one end side of the liquid medicine storage member 40 so that, with the application of a predetermined external pressure, the liquid medicine storage member 40 is easily opened thus allowing the liquid medicine 43 sealed in the liquid medicine storage member 40 to move to the air chamber 33. Further, the opening portion 41 is disposed near the applying portion 21 so that the liquid medicine 43, which is discharged from the inside of the liquid medicine storage member 40 due to the opening of the opening portion 41, can be effectively impregnated into the applying portion 21. A predetermined external pressure is applied to the liquid medicine storage member 40 before the applicator with a liquid medicine 10 is opened by peeling away the top film 32 or after the top film 32 is slightly peeled away to an extent that the inside and the outside of the applicator with a liquid medicine 10 are made to communicate with each other. With such an operation, the liquid medicine 43 sealed in the inside of the liquid medicine storage member 40 can be moved to the air chamber 33 without leaking to other portions.

The top film 32 also has point seal portions 51, which are point adhered, near the handle portion storage chamber 34 of the sheet portion 35 of the bottom film 31. The point seal portions 51 are seal portions which can be peeled by a predetermined external pressure. When the point seal portions 51 are peeled away, a volume between the sheet portion 35 and the top film 32 is increased as described later so that the cushioning effect caused by air sealed in the air chamber 33 can be reduced. Further, a portion of the top film 32 which opposedly faces the sheet portion 35 is adhered to the bottom film 31 only by means of the point seal portions 51, and other portions of the portion of the top film 32 are not adhered to the bottom film 31. As described above, the above-mentioned remaining portions are not adhered to the bottom film 31 so that a slight space, into which air in the air chamber 33 flows, is formed between the portion of the top film 32 which opposedly faces the sheet portion 35 and the sheet portion 35. Accordingly, during transport, storage or the like, the cushioning effect caused by air can be increased with respect to an accidental external pressure. Further, if an accidental strong external pressure is applied to the applicator with a liquid medicine 10 during transport, storage or the like, the point seal portions 51 are peeled away so that an impact to the liquid medicine storage member 40 can be reduced.

Further, assuming a maximum injection amount of air injected into the air chamber 33 as 100, an air injection amount, at which the cushioning effect caused by air can be acquired more reliably, is preferably 40 to 90, and more preferably 50 to 80. A volume of the air chamber 33 is defined as 100 when, for example, dimensions of respective sides of the air chamber 33 in the depth direction and in the width direction are set to dimensions which allow the applying portion 21 to be stored in the air chamber 33 with a certain surplus (being dimensions which are 110% to 140% of respective sides of the applying portion 21), and a dimension of the air chamber 33 in the length direction (the extending direction of the handle portion 22) is set to approximately half of a length of the handle portion 22.

Alternatively, by forming the air chamber 33 with a small depth so as to have the above-mentioned air injection amount, while the required cushioning effect caused by air is maintained during transport or the like, at the time of opening the liquid medicine storage member 40, the cushioning effect caused by air can be reduced. To be more specific, the air chamber 33 is formed on the one end side and the other end side, or the air chamber 33 and the handle portion storage chamber 34 are formed on the one end side and the other end side, and a predetermined amount of air is sealed in the air chamber 33 and the handle portion storage chamber 34. Accordingly, even when an external pressure is accidentally applied to the applicator with a liquid medicine 10 during transport, storage or the like, it is possible to prevent the liquid medicine storage member 40 from being opened due to the cushioning effect caused by air. At the same time, when the liquid medicine storage member 40 is opened, an external pressure can be directly and easily applied to the liquid medicine storage member 40 by performing an operation such as squeezing a portion corresponding to the liquid medicine storage member 40 so that the liquid medicine storage member 40 can be opened. Further, a configuration, where the air chamber is formed with a small depth whereby the cushioning effect caused by air is reduced, may be combined with a configuration where the point seal portions 51 are formed.

Further, the top film 32 has an outer peripheral seal portion 52 formed along an outer edge of the bottom film 31 so that the top film 32 and the bottom film 31 are adhered to each other. A strong seal portion 53 is formed on the one end side of the outer peripheral seal 52 so as to prevent the top film 32 and the bottom film 31 from being easily peeled away. It is preferable that a seal length of the strong seal portion 53 be set to 1 to 3 cm, for example. To allow the top film 32 and the bottom film 31 to be peeled away from each other, it is preferable that the outer peripheral seal 52 adhere the top film 32 and the bottom film 31 to each other with a width of approximately 1 to 10 mm. Alternatively, it is preferable that a plurality of outer peripheral seals 52 adhere the top film 32 and the bottom film 31 to each other with a width of approximately 1 to 2 mm.

Still further, an air removal portion 54 having a V shape may be formed on the other end side of the outer peripheral seal portion 52. With the formation of the air removal portion 54, when the air chamber 33 is pressed so as to peel away the point seal portions 51 as described later, air removal portion 54 ruptures substantially at or around the same time as the peeling of the point seal portions 51. Accordingly, air sealed in the air chamber 33 is released through the air removal portion 54 so that the cushioning effect described later can be reduced.

Any material may be used for forming the bottom film 31, the top film 32 and the liquid medicine storage member 40. For example, it is preferable to form the bottom film 31, the top film 32 and the liquid medicine storage member 40 using a film or a sheet made of a synthetic resin. To be more specific, polystyrene, polyethylene, polypropylene, polyethylene terephthalate or the like is preferably applicable. Further, each of the adhesive portions such as the point seal portions 51, the outer peripheral seal portion 52, the strong seal portion 53, the air removal portion 54, the opening portion 41 and the liquid medicine storage member sealing portion 42 adhere members to each other by heat welding.

Next, a method of using the applicator with a liquid medicine 10 according to this embodiment is described with reference to FIG. 4 to FIG. 8.

As shown in FIG. 4, the air chamber 33 is squeezed from the outside so as to apply an external pressure to an extent that the point seal portions 51 are peeled away by an air pressure in the air chamber. When the point seal portions 51 are peeled away, a space between the bottom film 31 and the top film 32 is increased so that an internal pressure of air sealed in the air chamber 33 is lowered whereby the cushioning effect caused by air is reduced.

As described above, when an external pressure is applied to the applicator with a liquid medicine 10 in a state where the cushioning effect caused by air sealed in the air chamber 33 is reduced, as shown in FIG. 5, the external pressure is directly applied to the liquid medicine storage member 40 so that the opening portion 41 is opened whereby the liquid medicine 43 leaks to the air chamber 33.

In regards of a structure for reducing the cushioning effect caused by air, in addition to the above-mentioned structure, a structure may be adopted where an air chamber with a smaller volume is provided thus reducing the cushioning effect caused by air at a portion of the liquid medicine storage member 40. Alternatively, a structure may be adopted where both the above-mentioned structures are used in combination thus reducing the cushioning effect caused by air. Still further, as described later, when the inside and the outside of the applicator with a liquid medicine 10 are made to communicate with each other through the air removal portion 54, the cushioning effect may be further reduced due to a configuration where air sealed in the applicator with a liquid medicine 10 leaks to the outside of the applicator with a liquid medicine 10 through the air removal portion 54.

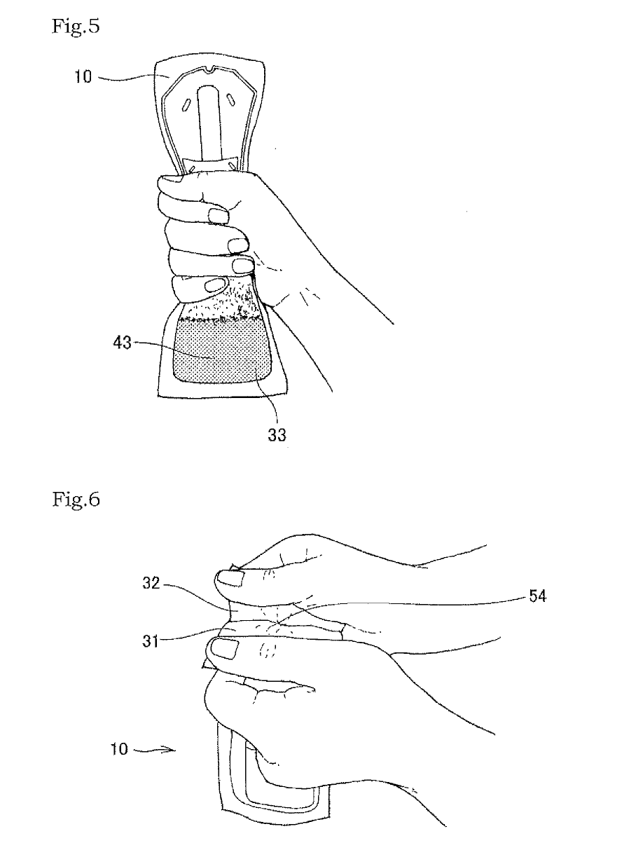

As shown in FIG. 6, to open the liquid medicine storage member 40 in a more reliable manner, the bottom film 31 and the other end side of the top film 32 are peeled away to an extent that the inside and the outside of the applicator with a liquid medicine 10 are made to communicate with each other. With such an operation, the liquid medicine storage member 40 can be opened more easily.

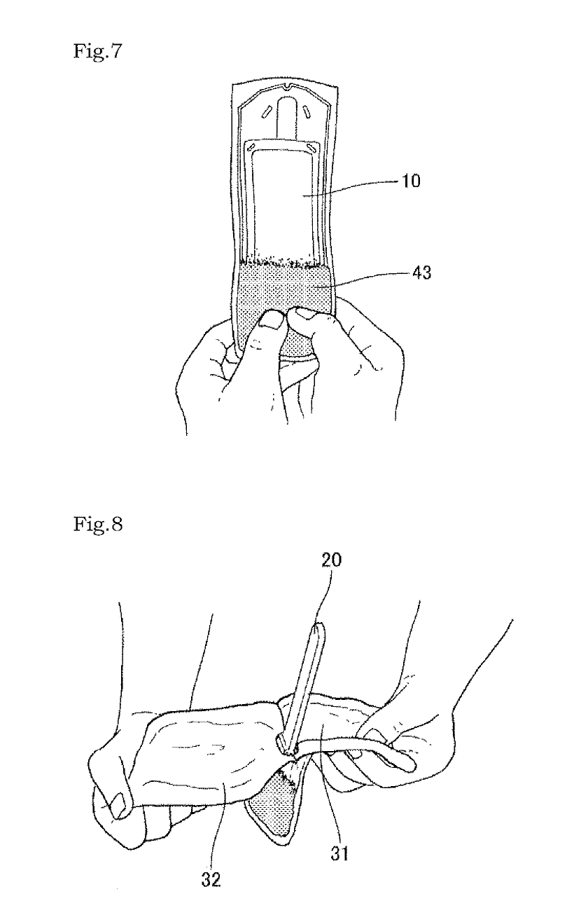

Further, when the liquid medicine 43 is held in the air chamber 33, the liquid medicine 43 is gradually impregnated into the applying portion 21. When impregnation of the liquid medicine 43 in the applying portion 21 is required to be accelerated so as to perform the operation quickly, as shown in FIG. 7, to allow the liquid medicine 43 to be sufficiently impregnated into the applying portion 21, the applying portion 21 is pressed to the liquid medicine storage member 40 and, then, is released from the liquid medicine storage member 40. By performing such a pressing operation, almost the whole amount of the liquid medicine 43 can be impregnated into the applying portion 21 more effectively.

Finally, as shown in FIG. 8, the liquid medicine 43 is sufficiently impregnated into the applying portion 21 and, thereafter, the top film 32 and the bottom film 31 are further peeled away and, then, the applying member 20 is extracted and used. At this time, the top film 32 and the bottom film 31 have the strong seal portion 53 on the one end side thereof so that the top film 32 and the bottom film 31 maintain the form of an integral body by way of the strong seal portion 53. As described above, the top film 32 is not completely peeled away from the bottom film 31 so that disposal of the storage member 20 is easily performed. In addition to the above, the liquid medicine 43, which is not impregnated into the applying portion 21 and remains in the air chamber 33, is held in the air chamber 33 without spreading to the outside.

With respect to the above-mentioned applicator with a liquid medicine according to this embodiment, the description has been made with respect a case where the top film and the bottom film are adhered to each other by heat welding. However, an adhesion method is not limited to the above-mentioned method. The top film and the bottom film may be adhered to each other using an adhesive agent. Provided that the top film and the bottom film are adhered to each other with predetermined adhesion strength, any of a variety of adhesion methods is applicable. Further, the description has been made with respect to the case where the liquid medicine storage member 40 has a bag shape, and the opening portion of the liquid medicine storage member 40 is formed of a weak seal having an approximately V shape. However, the liquid medicine storage member 40 may be formed of a container where a resin bottle is sealed using a weak seal. It is apparent from the description of Claims that the configurations which are obtained by adding such a change or modification also fall within the technical scope of the present invention.

### REFERENCE SIGNS LIST

10 applicator with liquid medicine,
20 applying member,
21 applying portion,
22 handle portion,
30 storage member,
31 bottom film,
32 top film,
33 air chamber,
34 handle portion storage chamber,
35 sheet portion,
40 liquid medicine storage member,
41 opening portion,
42 liquid medicine storage member sealing portion,
43 liquid medicine,
51 point seal portion,
52 outer peripheral seal portion,
53 strong seal portion,
54 air removal portion.

1. An applicator with a liquid medicine, the applicator comprising:
an applying member having a handle portion extending in a longitudinal direction and an applying portion mounted on one end of the handle portion and capable of being impregnated with a liquid medicine; and a storage member for storing the applying member, wherein
the storage member includes: a bottom film in which the applying member is stored; and a top film which closes the bottom film thus storing the applying member and a liquid medicine storage member, in which the liquid medicine is sealed, in the storage member, and
the bottom film has an air chamber which is capable of storing at least the applying portion and a predetermined amount of air.

2. The applicator with a liquid medicine according to claim 1, wherein
the bottom film has a handle portion storage chamber which is made to communicate with the air chamber and stores the other end side of the handle portion.

3. The applicator with a liquid medicine according to claim 1, wherein the liquid medicine storage member is mounted on the top film, and the top film has a point seal portion which is adhered to a portion near the handle portion storage chamber and is peelable.

4. The applicator with a liquid medicine according to claim 1, wherein the liquid medicine storage member is disposed on the top film along an extending direction of the handle portion, the other end side of the liquid medicine storage member being adhered to the top film, and an opening portion capable of being easily opened formed on one end side of the liquid medicine storage member.

5. The applicator with a liquid medicine according to claim 1, wherein the top film is adhered to the bottom film along an outer edge of the bottom film, and is adhered to the bottom film such that the top film is not easily peelable on said one end side of the bottom film.

6. The applicator with a liquid medicine according to claim 1, wherein the top film is adhered to the bottom film along the outer edge of the bottom film, and the top film is adhered to the other end side of the bottom film by way of an air removal portion which allows the top film to be easily peeled away from the bottom film.

7. The applicator with a liquid medicine according to claim 6, wherein the air removal portion is formed of a weak seal portion formed in an approximately V shape.
